(19) United States
(12) Patent Application Publication
(10) Pub. No.: US 2014/0039422 A1
(43) Pub. Date: Feb. 6, 2014

(54) FULL-BODY GARMENT OR SYSTEM OF GARMENTS FOR TREATMENT OF SKIN DISORDERS

(71) Applicant: AD RescueWear, LLC, Denver, CO (US)

(72) Inventor: Elizabeth Ann Scott, Denver, CO (US)

(73) Assignee: AD RESCUEWEAR, LLC, Denver, CO (US)

(21) Appl. No.: 13/744,257

(22) Filed: Jan. 17, 2013

Related U.S. Application Data
(60) Provisional application No. 61/587,579, filed on Jan. 17, 2012.

(51) Int. Cl. A61M 35/00

(52) U.S. Cl. CPC A61M 35/00
USPC 604/292; 604/293

(57) ABSTRACT

Aspects of the present disclosure disclose a garment for treatment of atopic dermatitis. In one aspect, the disclosure is directed a close fitting full body garment. In particular, the full body garment includes a torso covering portion, a pair of leg covering portions and a pair of arm covering portions. The pair of arm covering portions further include an integrated hand covering portions, and the pair of leg covering portions further include a pair of foot covering portions. The disclosed system is constructed from a fabric capable of moisture management that when in contact with the skin surface of the wearer provides an optimal relief from the symptoms of the skin disorder.

### CROSS REFERENCE TO RELATED APPLICATIONS

This application claims priority under 35 U.S.C. §119(e) to provisional application No. 61/587,579 titled “Full-Body Garment or System of Garments for Treatment of Skin Disorders” filed on Jan. 17, 2012, which is hereby incorporated by reference herein.

### FIELD OF THE DISCLOSURE

Aspects of the present disclosure relate generally to a garment or system of garments, intended particularly for infants and young children, in the treatment of atopic dermatitis.

### BACKGROUND

Atopic dermatitis, also called atopic eczema, is a common chronic skin disorder that is most prevalent among infants and young children, with 65% of children developing some symptoms before the age of one. Over the last three decades the occurrence of this skin condition has risen inexplicably. It is now estimated that currently between 10-15% of children in the Unites States are expected to experience this condition sometime during their lifetime, as compared to 7% around 1960.

Some of the symptoms experienced by individuals affected by atopic dermatitis include dry, itchy and easily irritated skin. The red itchy rash that is most commonly associated with this skin disorder can appear on any of the skin surfaces and in most cases causes individuals to scratch. Although the skin is already very sensitive to begin with, the scratching further irritates it, which in turn makes the skin even more sensitive and itchy, and leads to further scratching. This itch-scratch cycle in the end often leads to skin breakdown, infections and pain. Thus, the atopic dermatitis can have a significant impact on the quality of life of individuals and their families as it can interfere with both their daily activities and often makes it difficult to rest.

Depending on the severity of atopic dermatitis, treatment may include a simple recommendation not to scratch the affected areas, as well as application of over the counter or prescription creams and ointments. In the most severe cases, however, a wet wrap therapy treatment is recommended to alleviate the symptoms and help heal and protect the skin. It has been shown that the wet wrap therapy is effective in the treatment of atopic dermatitis. By keeping the skin moist, the inflammation and irritation of the skin can be significantly reduced, which in turn reduces scratching, and complications associated with it. Consequently, more restful nights and less interruption in daily activities is achieved.

In its current form, however, the wet wrap therapy usually calls for dressings that are made from bandages and/or articles of clothing. Typically, it involves application of a moisturizer or prescribed ointment or cream after a prolonged soaking in lukewarm water. In order to prevent rubbing off the emollients and to hold the moisture in the skin, wet bandages are applied against the skin. The process of wrapping bandages around various parts of the body, however, is both lengthy and uncomfortable. To begin with, it is very difficult to wrap the bandages around all the affected parts of the body uniformly, resulting in areas that are wrapped either too tightly or too loosely. When bandages are wrapped against the skin too tight, the normal blood flow to those parts of the body is prevented, making it very uncomfortable for the person wearing them. On the other hand, when bandages are fitting too loosely the effectiveness of the treatment deteriorates as the gap between the skin and the bandages makes it harder to keep the moisture in the skin. Also, the loosely fitting bandages can rub against already irritated skin, causing more irritation, and thus making the person uncomfortable.

Similarly, when using conventional clothing, like cotton pajamas for babies and toddlers, the garment is often too loose when soaked, has rough internal surfaces that irritate the skin and are difficult to put on or take off when wet. Furthermore, because conventional clothing or garments, such as for example ready to wear bandage type garments, do not include attached hand and foot covers, gloves, socks or bandages are typically used to cover hands and feet for treatment and to reduce scratching of the affected areas. However, because these foot and hand covers can be easily removed, the caregiver is often required to either tape or sew them to the separate garments (i.e. conventional clothing or garments), which further complicates the treatment process. Thus, the complexity associated with performing a wet wrap therapy in its current form and especially the multitude of separate items and steps that need to be used and completed to conduct the treatment often leads to the parents and caregivers being confused as to which type of clothing or pajamas to use resulting in the treatment being ineffective if the wrong type is used.

Thus, regardless of its benefits and effectiveness both the individuals and their caregivers find the wet wrap therapy process very cumbersome and as a result often fail to comply with treatment recommendations. Also, the fact that in its present form the wet wrap therapy is uncomfortable, further results in non-compliance. The complexity and time involved in performing the treatment is especially difficult in cases when the affected individual is a child for whom it is almost impossible to sit still for the amount of time required to complete all the steps of the treatment. Hence, there exists a need to simplify the wet-wrap therapy while at the same time making it more effective and comforting. In particular there is a need for a garment or a system of garments that would allow for an easy and convenient and comfortable way to comply with the requirements of the wet wrap therapy and provide comfort to its wearer.

### SUMMARY

Generally, embodiments of the present disclosure provide garments or a system of garments that can be used in the treatment of atopic dermatitis. As such, these ready-made garments or system of garments are intended to simplify the process of wet wrap therapy by meeting the special needs and provide comfort to the person undergoing treatment. One particular example of a garment of the present disclosure includes a full-body garment for treatment of atopic dermatitis in infants and young children made from a light weight hydrophilic fabric. In particular the garment is made from a fabric that is capable of conforming to a body of a wearer such that the garment remains in substantially direct contact with the skin surface of the wearer. Furthermore, the fabric used in the construction of the garment is capable of moisture management and is optimized for moisture absorption when the fabric is being wetted and for a prolonged moisture release when the fabric is placed against the surface of the skin. The garment of the present disclosure further includes a close fitting torso covering portion provided with a pair of close fitting extendable sleeves and a close fitting pair of leg covering portions. The extendable sleeves of the garment include an integrated fold-over hand coverings and a pair of leg covering portions include a pair of feet covering portions. In the middle of the close fitting torso covering portion is a longitudinal elongate opening that extends throughout substantially an entire length of the front of the torso covering portion between a neckline of the garment and terminating at around a crotch area.

Embodiments and advantages of the disclosure will be realized by those skilled in the art upon reading the detailed description of the present disclosure presented below.

### BRIEF DESCRIPTION OF THE DRAWINGS

FIG. 1 is a view of a garment in accordance with aspects of the current disclosure;

FIG. 2 is a view of a garment in accordance with other aspects of the current disclosure;

FIGS. 3A through 3E illustrate an extendable sleeve with a fold-over hand covering in accordance with aspects of present disclosure;

FIGS. 4 illustrate an extendable sleeve with a fold-over hand covering and an integrated wet wrap therapy glove in accordance with other aspects of the current disclosure;

FIGS. 5A and 5B illustrates foot covering in accordance with aspects of present disclosure;

FIG. 6A illustrates fastening means in accordance with aspects of present disclosure;

FIG. 6B illustrates fastening means in accordance with other aspects of present disclosure;

FIGS. 6C and 6D illustrate fastening means along with some other features in accordance with other aspects of present disclosure;

FIG. 6E illustrates some of the features of fastening means in accordance with aspects of present disclosure;

FIG. 6F illustrates some of the features of fastening means in accordance with other aspects of present disclosure;

FIGS. 7 illustrate seams used to connect various parts of the garment constructed in accordance with aspects of present disclosure;

FIG. 8 illustrates an example of a portion of a garment designed to cover only a specific body part that in accordance with aspects of present disclosure.

### DETAILED DESCRIPTION

The present disclosure is directed to a garment or a system of garments that make the treatment of atopic dermatitis or any other similar skin disorder more convenient and effective relative to conventional methods. In particular, the garment or system of garments, in accordance with the present disclosure, simplify the process of wet wrap therapy by providing a ready made or prefabricated garment that will make it easier to comply with a prescribed wet wrap therapy treatment, make the treatment more comfortable, and/or make the treatment more effective, among other advantages. Thus, some of the advantages of the garments or system of garments made in accordance with the current disclosure include the ease by which a parent may dress or undress a child being treated with the garment, the ability to treat affected areas uniformly and with greater wear comfort. In particular, some of these benefits are achieved through appropriate garment design and selection of suitable fabrics and sewing methods to construct the garments or system of garments.

In accordance with one specific embodiment of the disclosure there is provided a full-body garment adapted to cover the entire torso, arms, legs, hands and feet of a person being treated for atopic dermatitis, or any other similar skin disorder. This full-body garment may be made of one or more fabric layers or be comprised of a system of garments that can be used separately or in combination, depending on the need. In the case of a base garment alone or for use in a system, it is possible for the garment contacting the skin to be arranged to keep moisture in contact with the skin, minimize the number of seams and other surfaces that might irritate the skin, and provide mechanisms to efficiently dress using the garment. Some of these aims are in contravention to conventional garment design where it is often desirable to wick moisture away from the skin, or where designs optimize ease of dressing or undressing at the expense of more surfaces or features that might irritate very sensitive skin but would not provide any noticeable irritation to children or others without such sensitive skin. In a system, an outer garment or garments may be particularly suited to providing warmth and/or blocking moisture. It should be recognized that when treating a baby or toddler, the garment, which may be a base garment, is kept moist for effective treatment. Thus, the other garments are designed and/or layered, with the notion that the toddler will be wearing a wet garment. In the case that the child is wearing the base layer dry with emollients or to protect skin, the layered garments provide an added layer of warmth or protection from scratching.

Alternatively, in accordance with another embodiment of the current disclosure the garment can also be fashioned as a separate top and bottom with hand and foot covering portions. Also, separate coverings for specific body parts, that are most likely to be affected by any of the previously disclosed skin disorders are disclosed.

In one particular example, a primary layer or a first garment in a system of garments is a close fitting, hydrophilic, wet wrap layer or garment. One purpose of this layer or garment is to help mange skin moisture. In particular, the garment helps to regulate and restore water balance in the skin surface promoting healing. According to one embodiment, the garment may be designed such that moisture absorption is optimized when the garment is being wetted and moisture release is prolonged once the garment is placed in contact with the skin surface. In particular, keeping the moisture from the garment against the skin for an extended period of time allows for both the increase in the effectiveness of the treatment and also makes it more convenient to use since it provides for less frequent rewetting of the garment. This objective is in contrast with many conventional garments, particularly many performance garments, where the objective is wick moisture away form the skin, maximize the ability of the garment to dry when wet, repel moisture, and/or minimize absorption of moisture. Although the main purpose of the wet wrap garment is to be used in its wetted form, it should be recognized that the wet wrap garment can also serve as a barrier against scratching if worn wet or dry. As such, the chosen fabric and design serves a dual purpose. It allows to hold more water than conventional fabrics, such as for example cotton, when wet but remains soft when dry which is a bonus for sensitive skin. Furthermore, the wicking component of the fabric enables it to reduce bacteria growth when dry.

Another feature of this layer or first wet wrap garment is that it be body hugging or close fitting. That is, the garment material and design is such that there is little or no gap between the garment and the skin surface while the garment is worn. By being in a direct contact with the skin surface, the garment helps to improve the overall effectiveness of the wet wrap therapy for at least two reasons. First, by eliminating or reducing the gaps between the garment and the skin surface, any unnecessary rubbing between the garment and the skin surface is prevented, which results in less skin irritation. This is a significant advantage over the conventional treatment method in which the wet bandages applied against the skin often become loose, or a conventional garment, such as cotton pajamas, becomes baggy when wetted, leading to less effective skin contact and unnecessary skin irritation and loss of wear comfort, which diminish the effectiveness of the treatment. Second, by being in a uniform and direct contact with the skin surface, the close fitting of the wetted hydrophilic garment keeps a majority of the skin moist and delays moisture evaporation. Because the close fitting of the garment is an important feature of the garment made in accordance with the current disclosure, various sizes of garments are contemplated. For example the wet wrap garments can be made to fit infants from 0 to 24 months, as well as toddlers and older children in sizes 2T to 5T. Accordingly, selected size should provide for a close fitting of the garment.

It is yet another feature of the wet wrap garment that it is light weight and comfortable to wear against the skin. As such, although the fabric should have a good water retentive capability, it should not absorb water to the extent that the garment becomes too heavy and thus uncomfortable to wear. Also, even though the wet wrap garment needs to be close fitting, it should be made out of fabric that allows for both easy dressing and undressing and does not impede movement. Furthermore, because the skin of an individual affected by atopic dermatitis is very sensitive, in order to help prevent any unnecessary skin reaction, the fabric of the wrap garment may be non-toxic, dye-free, fragrance free, formaldehyde free, and possibly be made of natural and/or organic fibers that appear soft and smooth to the skin. Additionally, since the garment is primarily designed to be used in a wetted form, the fabric should be capable of preventing or inhibiting bacterial growth.

Thus, in accordance with one aspect of the present disclosure the wet wrap garment is made from a fabric that is light weight, has smooth and soft appearance, inhibits growth of bacteria, is capable of providing effective moisture management, and is capable of conforming to the body when both wet and dry. For example, one way to achieve this is to blend a small amount of Spandex™ or a similar elastic fabric to a given hydrophilic type of fabric, such as, for example; lyocell (e.g. TENCEL®/lyocell), organic or conventional cotton, bamboo, hemp or any other similar fabric or microfiber fabric or combinations thereof.

Although, the wet wrap garment can be made solely of a water retentive fabric, a wet wrap garment made of fabric that includes integrated hydrophilic and hydrophobic layers is also contemplated. Thus, in another embodiment of the current disclosure, there is provided a close fitting garment in which a primary hydrophilic layer is provided on the inside of the garment, and a second hydrophobic layer on the outside of the garment. The hydrophobic layer may also be provided by a separate garment in a system of garments. By being on the inside of the garment, the primary hydrophilic or water retentive layer is in direct contact with the skin surface as described above, thus keeping the skin moist. The hydrophobic layer, which is on the outside of the garment, helps to keep the moisture in the hydrophilic layer and on the skin, reduces the amount of water being taken out from the primary layer and reduces evaporation. Also, the hydrophobic layer helps to prevent seeping of the moisture from the inside layer that often causes wetting of any other garments donned on the wet wrap garment or bedding. Thus, the garment of the current embodiment not only further increases the effectiveness of the treatment by helping to keep the moisture in the inner hydrophilic layer, but makes it more comfortable for the wearer as it prevents wetting of any other outer garment, bedding, or the like.

In accordance with yet another embodiment of the current disclosure, a wet wrap garment made of fabric that includes integrated water absorbing, water repelling, and thermal-insulation layers is provided. One of the advantages of the garment of the present embodiment, in which all layers are integrated into one garment, with the hydrophobic layer being sandwiched between the primary layer and the insulation layer, includes simplicity of use, as only one garment needs to be placed on a child to conduct the wet wrap therapy treatment.

Similarly, in a system of garments in accordance with another embodiment of the present disclosure, there is disclosed a second garment, such as a dry wrap garment that is fashioned in essentially the same manner as the wet wrap garment. This second garment is made from a mid-weight, hydrophobic fabric and has a roomier fit. The purpose of the dry wrap garment is to help retain moisture in the wet wrap garment and to help prevent seeping of water from the wet wrap garment to any other outer garments, bedding or the like. Accordingly, it is made from a water resistant or water repelling material. For example, a poly or nylon type fabric such as Supplex® that can be finished with a soft finish can be used. Also, because the dry wrap garment is typically not placed directly against the skin surface, but rather on top of the wet wrap garment, it is made somewhat larger than the primary wet wrap garment in order to provide ample space to accommodate the primary wet wrap garment. The dry wrap garment in accordance with the current embodiment, thus, not only makes the treatment effective by helping to retain more moisture in the wet wrap garment, but it also makes the treatment comfortable by preventing wetting of outer garments, bedding, etc. In addition, it should be understood that this garment is also designed for maximum comfort for sensitive skin and thus the fabric used to make the second garment may be a smooth and soft feeling fabric made from man made or natural fibers that are non-toxic and sustainable and/or organic materials.

In yet another embodiment of the present disclosure, the dry wrap layer or garment can be made of a fabric that includes an integrated water resistant inner lining or layer, and a thermal insulation outer layer. One way to construct the garment according to the current embodiment is, for example, to sew a water resistant inner lining to the outer thermal insulation layer. In the dry wrap layer or garment in accordance with the current embodiment the function of the lightweight inner lining is to prevent the seeping out of the moisture from the wet-wrap layer or garment, and the function of the thermal insulation outer layer of the dry wrap garment is to keep the child warm. Accordingly, the water resistant inner lining can be made from, for example, a poly or nylon type fabric such as Supplex® and the thermal insulation outer layer can be made from, for example, a medium weight natural or man made materials such as cotton or fleece or any other similar type of fabric. As such, the dry wrap garment made from the integrated water repelling layer and the thermal insulation layer provides for a simplified garment or system of garments.

In accordance with another embodiment of the present disclosure, a system of garments may include a third, thermal insulation layer or garment. One of the functions of the thermal insulation layer or garment is to provide warm insulation and to help maintain a stable body temperature. Thus, the fabric selected for this layer should be capable of limiting heat loss from the body, while maintaining freedom of movement and providing wear comfort. Because this tertiary thermal insulation layer or garment is typically placed on top of both the primary wet wrap garment, and the secondary dry wrap garment, it is sized such that it can be placed comfortably over the previous two layers or garments. However, in order not to degrade the added benefit of thermal insulation provided by warming up of the layer of air present between the thermal insulation layer or garment and the layer or garment placed immediately prior to the thermal insulation layer or garment, the gap formed between the two garments resulting from differences in sizing should be optimized to obtain the greatest thermal insulation benefits. Furthermore, the fabric selected for this layer should not only be capable of providing thermal insulation, but be light weight and comfortable. For example, the thermal insulation garment may be made of natural or man made materials such as cotton or fleece. However, any other fabric capable of performing the above specified function can be used. Also, in those instances when the thermal insulation layer comes in contact with any exposed skin surfaces, such as face or hands, the fabric selected for the thermal insulation layer or garment should be made from a soft fabric to help reduce scratching or skin irritation.

In another embodiment of the current disclosure, a system of garments made from three different layers or garments described above is contemplated. Such system of garments may include a first close fitting wet wrap garment, a second dry wrap garment and a third thermal insulation garment. In this three garment system, it is the function of the first garment to retain moisture to keep the skin moist, it is the function of the second garment to prevent seeping out of the water from the first garment, and it is the function of the third garment to help maintain body temperature at a comfortable level, as well as help prevent any unnecessary skin irritation if fabric comes into contact with any exposed skin surfaces. Furthermore, each garment of this three garment system can be put on separately or used in various combinations.

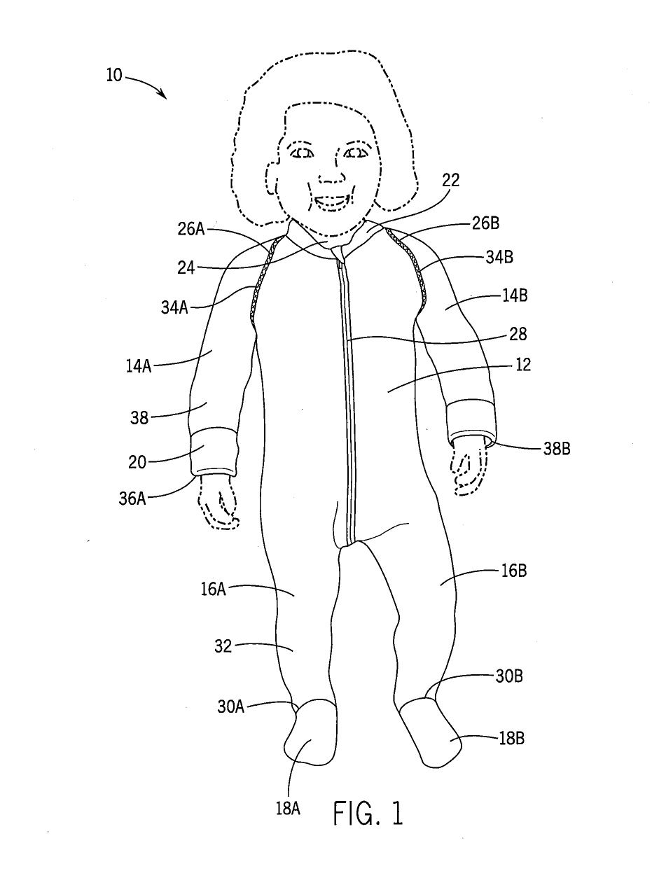

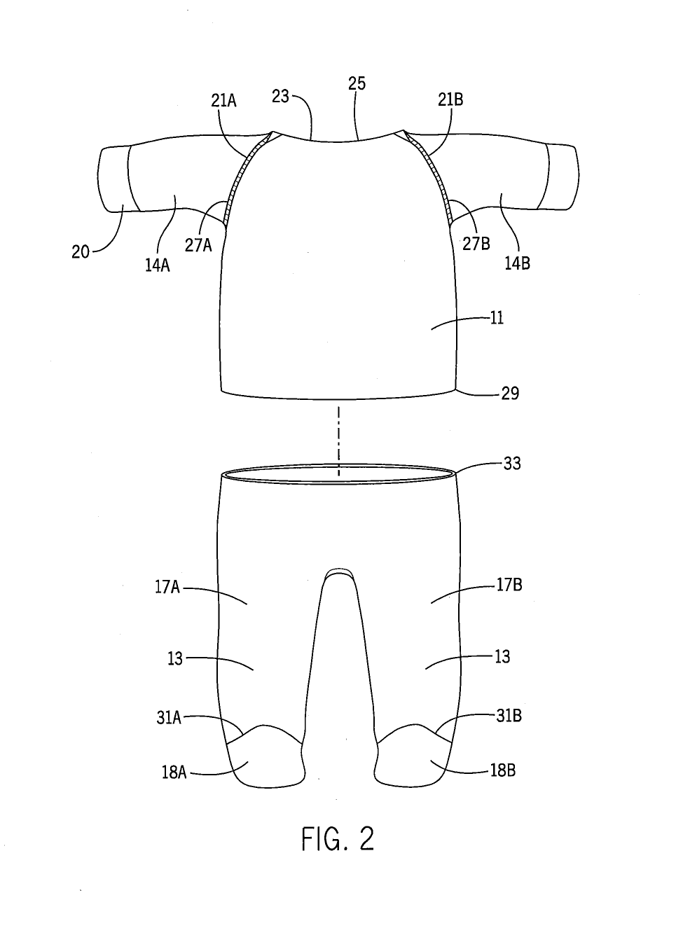

Referring now to the drawings, a body garment designed to improve the effectiveness of wet wrap therapy while providing comfort and convenience of use is shown in FIGS. 1 and 2. Generally, the disclosed body garment is designed to cover any or all of the body areas affected by any of the previously disclosed skin condition. Thus, the body garment 10 is shown with torso, arms, legs, hands, and feet covering portions. The body garment 10 is preferably close fitting and is made of fabric that helps to increase the effectiveness of the wet wrap therapy.

As shown in FIG. 1 the body garment 10, includes a torso covering portion 12 provided with a pair of arm covering portions or extendable sleeves 14A, 14B, a pair of leg covering portions 16A, 16B, and a collar 22. The torso covering portion has a neck opening 24, a pair of arm openings 26A, 26B, and a longitudinal elongate opening 28 in the middle of the torso covering portion. Although not shown in the drawings, in place of the elongate opening 28, the torso covering portion may alternatively include elongate and substantially diagonal openings along or proximate to the junctions at which the arm covering portions are attached to the torso covering portion. The openings may be closed with zippers, snaps, or other closure means as discussed herein relative to other embodiments. Thus, in this particular example, in order to dress a child in the garment, the sleeves may be opened at the shoulder and the entire upper front half of the garment may be folded down. In so doing, the upper half of the garment is opened about midway between the neck and waist in a line between the bottom of the underarms. The garment may then be fit to the child through this enlarged top opening. In this arrangement, it is possible to have less total zipper or snap seam length as compared to a garment that has a single long opening between the neck and waist, or lower (sum of the two shorter shoulder openings being less than the length of the longer opening along the torso) and thus less features to irritate the child's skin.

The leg covering portions, which are defined by a bottom ends 30A, 30B, an outer surface 32 and an inner surface (not shown) are essentially tubular, and are integrated with the torso covering portion 12. At the bottom end 30A, 30B of each leg covering portion 16A, 16B is an opening to which a foot enclosure or foot covering 18A, 18B is attached. The extendable sleeves are made of a fabric sewn into a tubular form, having an upper edge 34A, 34B, a lower edge 36A, 36B, an inner surface (not shown) and an outer surface 38. The upper edge 34A, 34B of each sleeve is attached to the torso covering portion at the respective arm opening 26A, 26B in the torso covering portion. Distal the torso connection, is an integrated, fold-over hand covering 20.

The exemplary garment shown in FIG. 1 includes several features that make it particular suitable for treatment of various skin disorders that may not otherwise be found in more conventional garments. One specific feature shown in FIG. 1 relates to garment surface uniformity. In particular, the garment according to the current disclosure is designed to minimize possible areas of irritation that may be created when various parts of the garment are joined together or when two separate garments for covering a lower and an upper body are used. Also, for the areas that necessarily need to joined together unperceivable seams may be used to eliminate any unevenness in the surface of the garment that is to be in contact with the skin surface. Furthermore, the design of the longitudinal elongate opening in the middle of the torso covering portions or alternatively the design of the elongate and substantially diagonal opening(s) at the junction(s) between the torso and arm coving potions, along with the closure means may be optimized to achieve a balance between garment surface uniformity and easiness of garment use. Thus, for example, the length of the longitudinal opening or the diagonal opening(s) may be selected to enable placement and removal of the garment with relative ease but simultaneously maximize surface uniformity. Moreover, additional pieces of fabric may be used to prevent certain parts of the garment from coming into contact with the skin surface. In particular, a piece of fabric for covering of the closure means along its length and ends might be used.

In FIG. 2, the body garment 10 in accordance with another embodiment of the current disclosure is shown. In this example, the body garment is fabricated with a separate top 11 and bottom 13 covering portions. According to this embodiment, the top covering portion 11 includes a neck opening 25 at the top end 23 of the top covering portion, a pair of arm openings 27A, 27B and a bottom end 29. A pair of arm covering portions or extendable sleeves, 14A, 14B are attached at the respective arm openings 27A, 27B of the top covering portion. The top covering portion may also include a longitudinal elongate opening (not shown) in the middle of the front or back of the torso covering portion that extends through at least a part of the top covering portion. Alternatively, an opening or openings extending along one or both sides of the torso covering portion, from about the side of the neck opening to about the shoulder area, as well as opening or openings 21A, 21 B extending along and/or proximate to the junctions at which the arm covering portions are attached to the torso covering portions of the garment, as discussed above relative to the full body garment, may be used. The bottom covering portion 13 is defined by a top end 33 and a pair of leg covering portions 17A, 17B. At the bottom ends 31A, 31 B of each leg covering portion is an opening to which a foot enclosure or foot covering 18A, 18B is attached. The top end 33 may include an elastic inside the waistband, or any other type of waistband that allows for a comfortable wear of the bottom covering portions can be used.

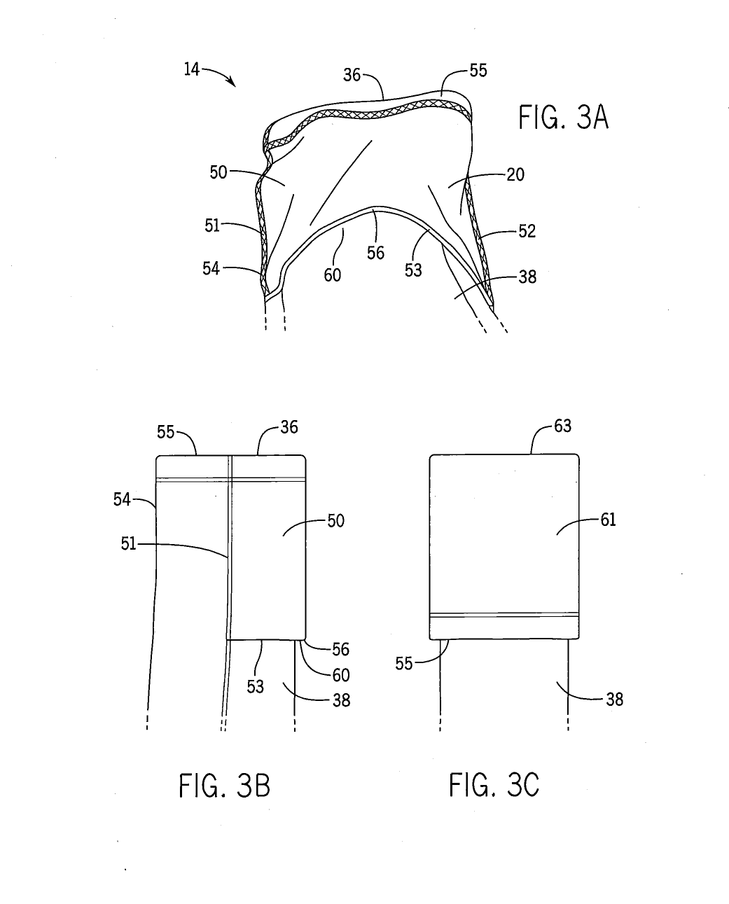

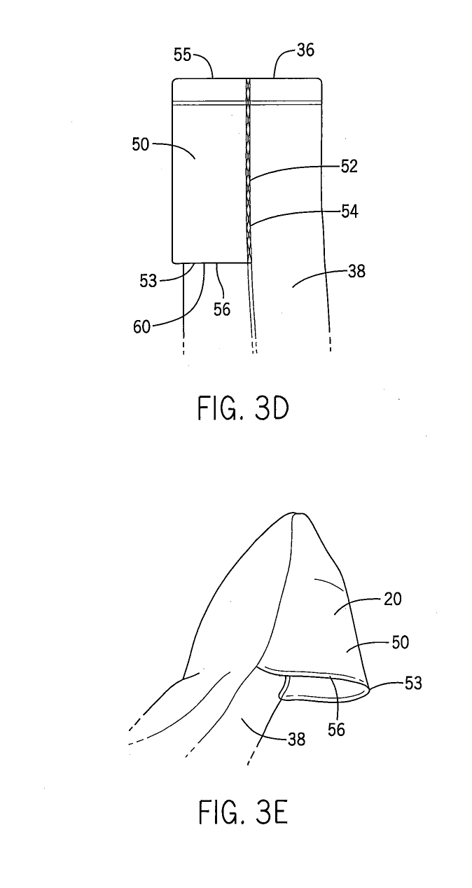

FIGS. 3A through 3E illustrate an extendable sleeve 14 with an integrated fold-over hand covering 20. This feature may be included in the embodiment depicted in FIG. 1, FIG. 2, or others. The extendable sleeve with the integrated fold-over hand covering 20 is designed to prevent the child from scratching, and/or provide wet wrap therapy to the hand and fingers. In one configuration, shown in FIGS. 3A, 3B and 3D the extendable sleeve extends to about the tips of the fingers when unfolded. Thus, while not shown, the wearer's finger tips may extend outward from the top of the sleeve. In order to provide access to the child's hand and/or a wet wrap therapy glove if used, the extendable sleeve can be folded back as shown in FIGS. 3C. In particular, when folded back, the extendable sleeve can form a cuff 61, which has a length that is approximately equal to the length of a hand with a folded edge 63 being proximate to the typical wearer's wrist area. Alternatively, the extendable sleeve can be simply pushed back up along the hand until the edge of the sleeve is aligned with the wrist of the wearer.

As shown in FIGS. 3A, 3B and 3D the extendable sleeve in the unfolded configuration includes a cuff assembly having a foldable cuff panel 50. In particular, according to one embodiment, the cuff panel may be formed from a rectangularly shaped piece of fabric, having a first and a second longitudinal edge 51, 52 and a first and a second lateral edge 53, 55, with one of the lateral edges being attached to the outer edge 36 of the outside surface 38 of the extendable sleeve, and the second lateral edge being a free edge longitudinally displaced from the first lateral edge. The rectangular piece of fabric being also sewn thereon along the longitudinal edges 51, 52 extending along the extendable sleeve on either the palmar or dorsal side of the hand. By attaching the rectangular piece of to the extendable sleeve only along a portion of the lower edge 36 of the sleeve or half of the circumference of the sleeve and along two sides of the extendable sleeve, a partial cuff or a pocket 60 is formed. In other words, because the cuff formed by the cuff panel does not extend along the full circumference of the lower edge 36 of the sleeve, the pocket on the outside surface 38 of the extendable sleeve is formed, which can be folded over the fingers when needed to provide a fold-over hand covering 20, as shown in FIG. 3E. Stated differently, the unattached or free edge 56 of the rectangular piece of fabric can be pulled or folded over the fingers in order to cover them. Since the main purpose of the fold-over hand covering is to prevent scratching, it does not necessarily need to be close fitting. This roomier fit provides for an easier placement of the fold-over hand covering over the hand if used in combination with the wet wrap therapy glove and allows the child to freely move their fingers inside the covering even if the wet therapy glove is not used. Also, without the wet wrap therapy glove, the fold-over hand covering may still provide wet wrap therapy albeit somewhat less effective than a glove.

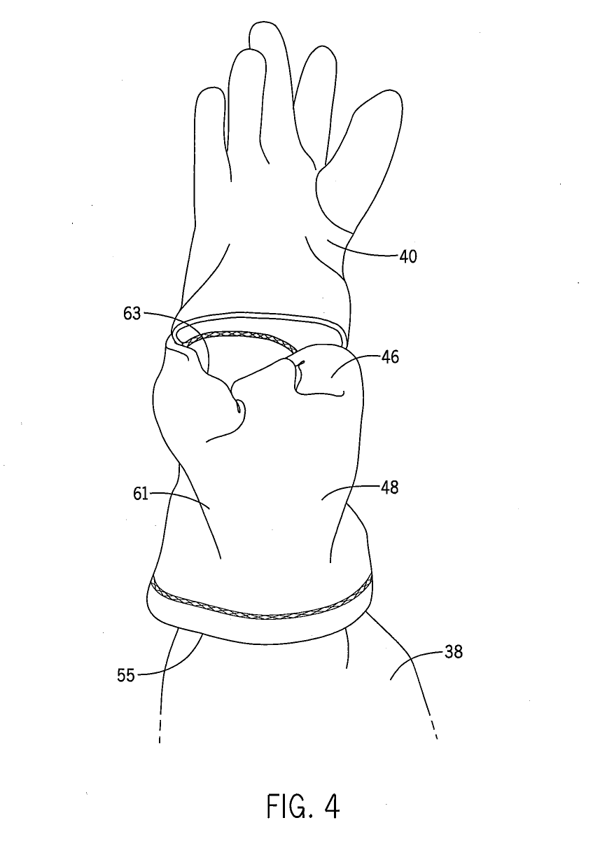

In FIG. 4, an implementation of a wet wrap garment with an integrated wet wrap therapy glove 40, that is intended to be placed over the bare hand prior to the placement of the fold-over hand covering 20 is shown. As shown in the figure, the wet wrap therapy glove 40 is attached to the inside surface 48 of the extendable sleeve, along a folded edge 46. More specifically, the glove 40 is attached on the inside surface of the extendable sleeve, on the dorsal side of the hand along the wrist area. By attaching the glove to the inside surface of the extendable sleeve, the extendable sleeve can be unfolded once the glove is placed over the bare hand, which in turn allows for later placement of the fold-over hand covering over the glove. Alternatively, the glove can be attached to the sleeve along the full circumferential length of the sleeve or be integral with the sleeve allowing to further increase the uniformity in the surface area of the garment . As described above with respect to the overall garment, in order to make the treatment of the hand more effective the glove should be close fitting. As such, it is contemplated that the glove will be made of different sizes that would match the overall size of the garment and provide for a close fitting of the glove against the skin surface. Furthermore, because the glove is intended to be in direct contact with the skin, it should be made from a water absorbent fabric and should be comfortable to wear against the skin. Accordingly, the glove can be made from the same fabric from which the remaining parts of the garment are made. Also, another example of fabric that is capable of performing the above described functions is 100% cotton. Alternatively, when a system of garments is used, a wet therapy glove can be included with the wet wrap garment and the integrated fold over covering can be included with the dry wrap garment.

Referring again to FIGS. 3A, 3B and 3D, according to one embodiment, the cuff panel may be connected to the extendable sleeve with seams 54 being on the outside surface 38 of the extendable sleeve when the cuff panel is in the unfolded configuration or the hand covering is not placed over the hand. Placing all of the seams 54 on the outside when the cuff panel is not folded over a hand or in use provides a hand covering with an overall smoother surface when the hand covering is eventually used, as shown in FIG. 3E. In other words, once the hand covering is placed over the hand, all of the seems used to connect the foldable cuff panel 50 to the outside surface of the extendable sleeve are on the inside of the hand covering which helps to prevent any irritation that may result from the seams coming into contact with any exposed body parts, such as, for example, face, when the garment is in use.

According to another embodiment, shown in FIGS. 3A, 3B and 3D, when a rectangulalry shaped piece of fabric is used as a cuff panel, three different types of seams may be used to attach the rectangularly shaped piece of fabric to the outside surface of the extendable sleeve. For example, a straight stitch 51 can be used to attach one of the longitudinal edges of the rectangular piece of fabric to the extendable sleeve, a flatlock seam 52 can be used to attach the second one of the longitudinal edges of the rectangular piece of fabric to the extendable sleeve, and a coverstitch can be used to attach one of the lateral edges 55 of the rectangular piece of fabric to the extendable sleeve. Furthermore, the unattached portion or the free edge of the rectangular piece of fabric can be hemmed with a seam that is on the inside of the pocket or partial cuff 60. In other words, the stitching used to hem the fabric is flat on the outside of the pocket when the fold-over hand covering is not placed over the hand.

Alternatively, the rectangularly shaped cuff panel can be formed from a fabric that has a width that is approximately the width of the hand and the length that is approximately twice the length of the hand. According to this embodiment, the rectangularly shaped fabric can be folded in half along its length, to provide a double layered cuff panel. Folding the rectangular piece of fabric over in the middle eliminates a need for hemming the free edge the fabric since the folded edge 56 serves as the edge finishing. According to another embodiment, all the seams used to attach and/or hem the rectangular piece of fabric 50 can be made on the inside or outside respectively or any of the sewing techniques, discussed below can be used in order to provide a greater wear comfort.

Although, the fold-over hand covering is described as being made of a rectangular piece of fabric, different shapes that would allow the fold-over hand covering to conform more to the general shape of the hand are also contemplated. Furthermore, the fabric used to make the hand-covering can be made of fabric that is different than that used for other portions of the garment.

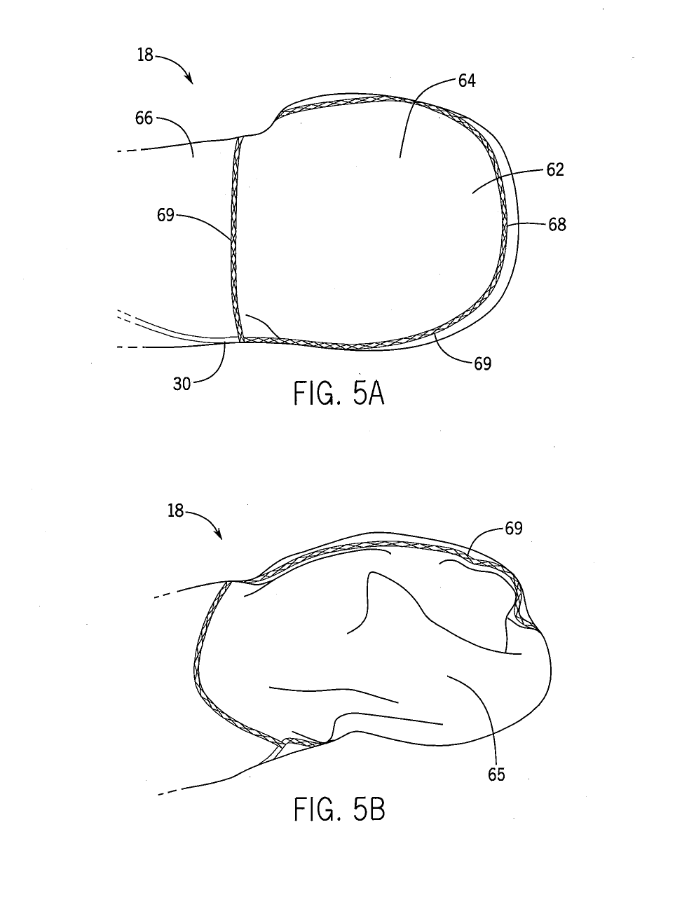

FIGS. 5A and 5B illustrate a foot covering or foot enclosure in accordance with one embodiment of the present disclosure. As shown in FIGS. 5A and 5B, the foot enclosure 18 is attached to the bottom or distal end 30 of the leg covering portion of the garment. The foot enclosure 18 includes a toe covering portion 62, an instep covering portion 64 and a sole covering portion 65, shown in FIG. 5B. According to one embodiment, the toe covering portion 62 and the instep covering portion 64 are formed from a single piece of fabric that is preferably connected to distal ends 30 of the garment and to the sole covering portions by stitching, with seams 69 running along an ankle area 66 and along the outer parameter 68 of the foot. According to one embodiment, all seams are placed on the outside of the foot covering in order to prevent skin irritation and provide comfort. Alternatively, it is contemplated that the foot covering will be attached to the bottom part of the leg seamlessly using a v-bed flat knitting machine or flatlock seam.

Also, the disclosed foot covering is designed such that it is close fitting and comfortable to wear against the skin. In particular, the foot covering is configured such that it is in direct contact with all the discrete parts of the foot, such as the toes, the instep, the ball, the arch and the heel. Alternatively, the foot covering may also include covering for each individual toe, for those instances when toes also need to be treated. Although the foot covering is designed to be closed fitting in order to improve the effectiveness of the wet wrap therapy, it is at the same time not constrictive and thus comfortable to wear. Thus, the foot covering, similarly to the remaining of the garment, comes in various sizes that either match the size of the garment or more closely match the size of the individual's foot and may include a non-slip material at the bottom surface of the foot covering.

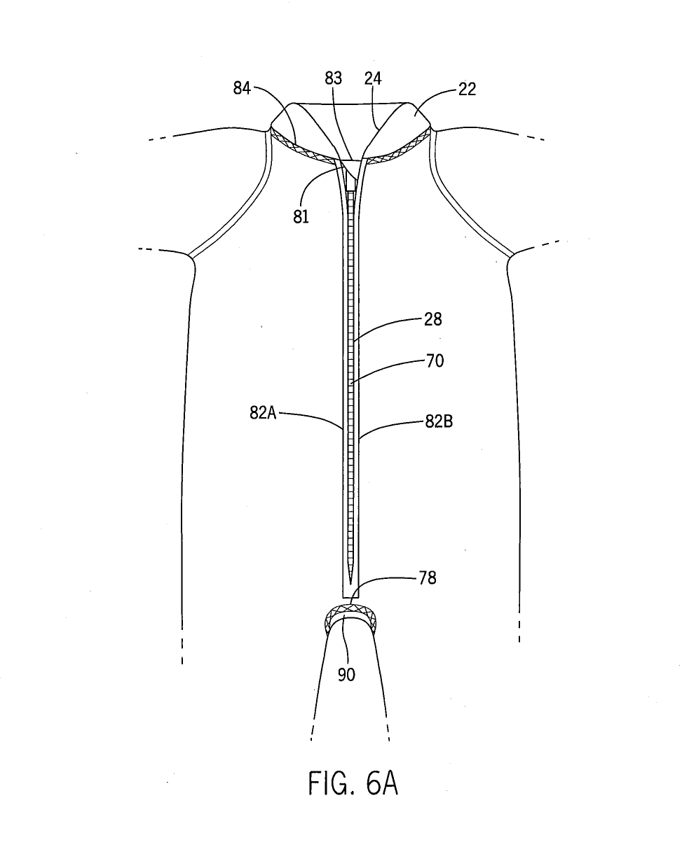

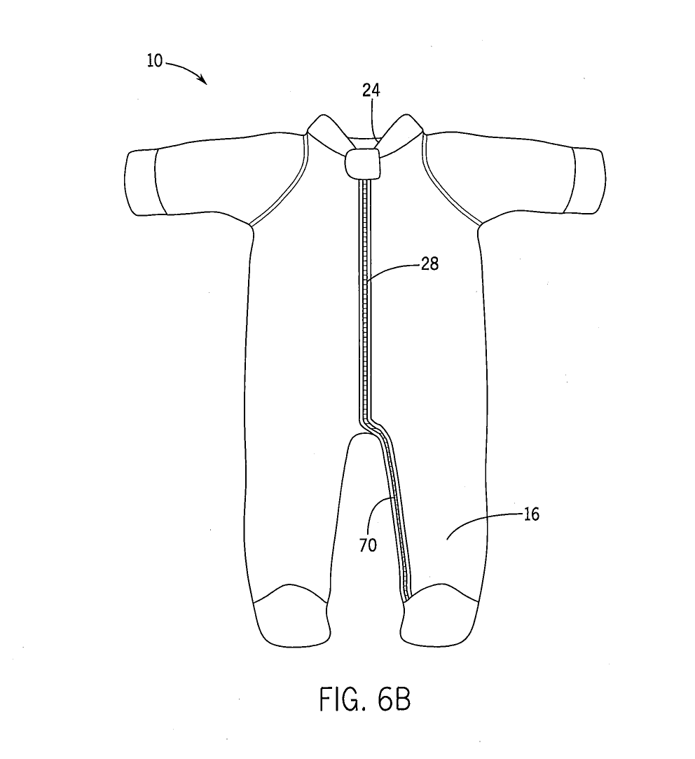

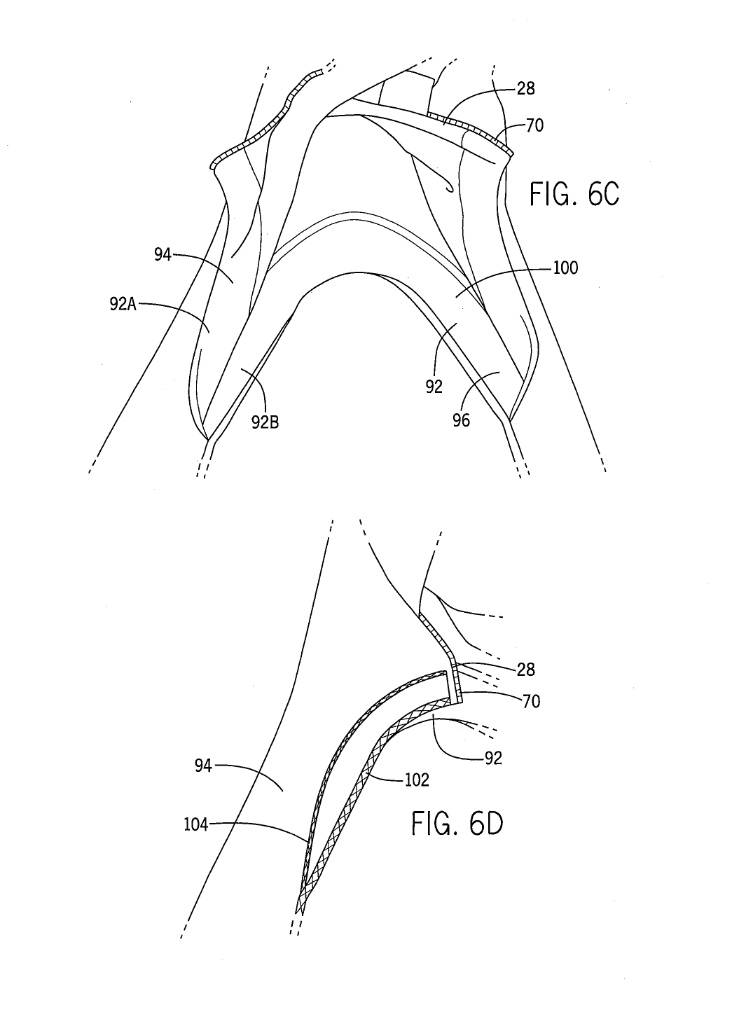

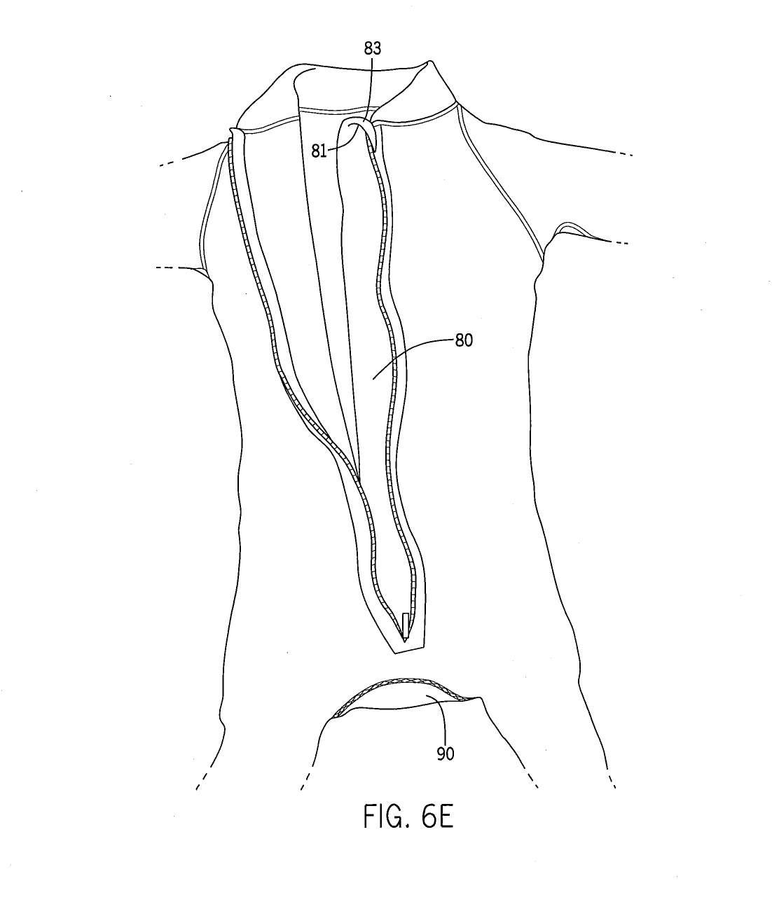

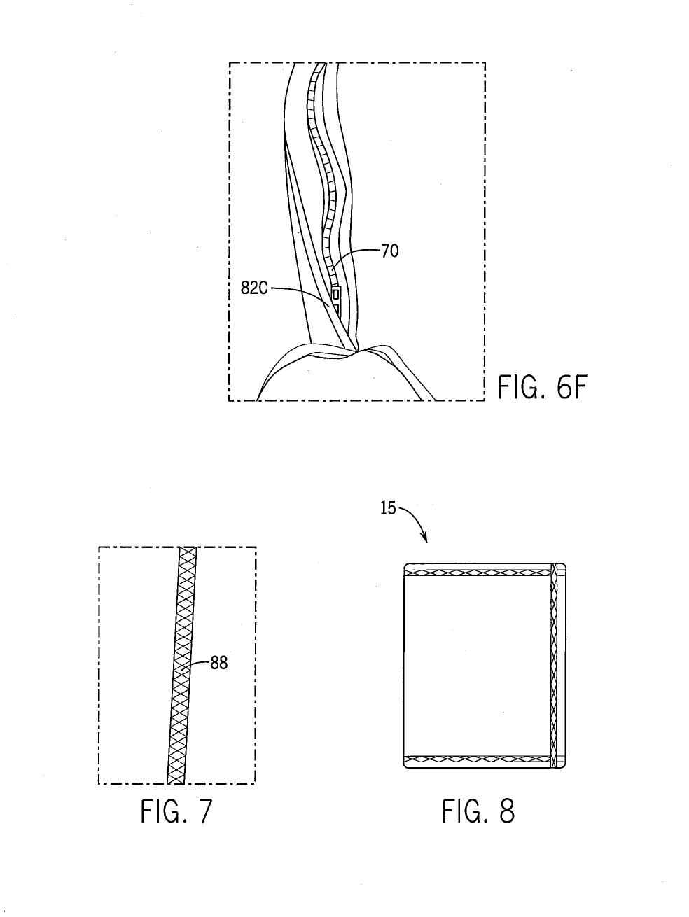

FIGS. 6A through 6F illustrate another feature of the present disclosure, which generally relates to the ease of dressing the child with the garment or removing the garment, in balance with enhancing effectiveness of the treatment and minimizing irritation that might be caused by seams, zippers or other features. Because the garment will often be used to treat infants and young children, it is designed such that dressing and undressing can be done efficiently and without much struggle. Also, the design provides for an easy diaper change when necessary. Thus, a longitudinal elongate opening 28, with a closure means 70 is provided in the middle of the torso portion of the garment. Alternatively, as discussed previously, elongate and substantially diagonal openings along or proximate to the junction(s) at which the arm covering portions are attached to the torso covering portions along with closure means may be provided. The closure means can be a zipper, or alternatively any other conventional closure means such as snaps, or the like that allow for closing and opening of the opening 28.

In many conventional full body children's garments the opening may extend through an entire length of the garment in order to optimize access to the diaper and dressing and undressing the child. For example, it is relatively easier to change a diaper when the opening extends from the neckline down the leg as the resulting opening in the garment provides greater access to the diaper and provides more room to move the garment away from the child when removing a soiled diaper. In the preset design, however, the opening does not extend so low to strike a balance between access, ease of dressing, and minimizing features that might irritate the skin or provide less effective skin contact and therefore less effective treatment. In other words, the opening does not extend down the leg in order to increase the treatment surface area and prevent access for scratching at skin. Similarly, when used in the separate top covering portion in a garment fabricated with a separate top and bottom covering portions, an opening that extends only through a portion of the top covering portion may be used. For example, an opening that extends from the neck opening to the middle of the torso may be provided. Alternatively, when elongate and substantially diagonal openings along or proximate to the junctions at which the arm covering portions are attached to the torso covering portions are used, the openings may extend only, for example, half way down the junction length.

More particularly, as shown in FIGS. 6A, a zipper that begins at the neck opening 24 and extends down to around the crotch area 78, is used. In particular, this configuration is designed to minimize areas of the garment that could potentially cause unnecessary skin irritation when coming in contact with the skin surface. More specifically, minimizing of the zipper length helps to increase uniformity of the total surface area of the garment thereby reducing potential for skin irritation due to the irregularities caused by the difference in the structure of the fabric used to construct the garment and the closure means and/or areas of attachment of the closure means. The zipper may open from the neck opening down to around the crotch area or alternatively, a reverse zipper configuration, such as a zipper that zips from bottom of the garment up (i.e. from the crotch area to the neck opening) may also be used.

As shown in FIG. 6B, a reverse zipper configuration may be used with the zipper extending from the bottom end of at least one of the leg covering portions 16 or from the crotch area up to the neck opening 24. More specifically, according to this embodiment, the zipper runs along the inside edge of one of the leg covering portions and through the longitudinal elongate opening 28 in the middle of the torso portion of the garment. The disclosed reverse zipper configuration provides for an easy and more comfortable diaper change in younger children and infants, since only the bottom part of the garment needs to be opened to access diaper area. In other words, when changing a diaper, a child can be kept warm and comfortable as only the bottom part of the garment, sufficient to allow for a diaper change needs to be opened up. Furthermore, when the garment needs to be put on the child or taken off, the zipper can be fully opened to provide a large access area to place the garment on the child or to take the garment off.

In accordance with yet another embodiment, a zipper or any other closure means can be also incorporated into the separate top and bottom design as discussed with reference to FIG. 2. In other words, a separate closure means can be included in the top covering portions, as well as in the bottom covering potion in order to facilitate placement and removal of each of the separate portions of the garment. Alternatively, the torso covering portion in the garment fashioned with separate top and bottom can be constructed such that no longitudinal opening or closure means is used. In this configuration, the garment is designed to be pulled over the child's head in order to cover the torso. Similarly, the leg covering portions can be fashioned so that no opening or closure means are used along any parts of the leg covering portions.

According to yet another embodiment of the current disclosure, shown in FIGS. 6C and 6D, the garment may be designed to include an opening 92 in around the crotch area of the garment. The opening may be particularly useful when the garment is used for infants or small children that require frequent diaper changes as only the needed parts of the crotch area and the lower part of the torso covering portions can be opened during the diaper change process allowing for greater comfort for the child. More specifically, as shown in FIGS. 6C and 6D the opening in the garment may extend from about half way down the inside of one of the leg covering portions, to about half way down the inside of the second leg covering portions, such that an opening in the shape of an arch is formed around the crotch area. In other words, the opening is formed by not sewing together the inside edges 93A, 93B of the front 94 and the back 96 sides of the integrated leg covering portions around the crotch area.

When used together with the reverse zipper 70 for closing the longitudinal elongate opening 28 that begins at the lower front part of the torso covering portion, the opening may be enlarged by pulling back to the sides the lower front parts of the torso covering portion of the garment and the upper front portion of the leg covering portion in order facilitate diaper changes. More specifically, according to this embodiment, both the front side 94 and the back side 96 of the integrated leg coverings of the garment, in around the crotch area, are cut out in a form of an arch, with the longitudinal elongate opening 28 cutting through the middle of the arch on the front side of the garment. As such, both the lower part of the torso covering portion, as well as the upper part of the leg covering portion can be pulled to the sides providing greater access to diaper area. Also, in one implementation a closing means such as snaps or the like, may be used to join the front and back of the integrated leg covering portions along the inside edges, around the crotch area.

Furthermore, the reinforcement areas may be provided along the inside edges 93A, 93B, of the leg covering portions. In particular, additional pieces of fabric 100, that are essentially shaped like an arch are attached close to the inside edges of the leg covering portions around the crotch area. The additional pieces of fabric extend half way down one leg covering portion, and run along the crotch area to half way down the other leg covering portion. The additional pieces of fabric are sewn to the inside edges of the front and back sides of the leg covering portions along two lines. First, a seam 102 is stitched along the inside edges of the leg covering portions in order to connect the additional pieces of fabric to the inside edges. A second seam 104 is stitched away from the edges in order to attach the additional piece of fabric to the garment. It should be noted that the seam which is away from the edge of the inside of the leg covering portions runs along the crotch area in a curvilinear manner. More specifically, the stitches begin at the inside edge of one of the leg covering portions and then run away from the edge and around the crotch area to the inside edge of the other leg covering portions.

Another aspect of the current disclosure shown in FIGS. 6A, 6E and 6F relates to the concept of minimizing skin irritation that could result from the closure means rubbing or coming into contact with any of the skin surfaces. In particular, coverings for both the inside and outside surfaces of the closure means are disclosed. As shown in FIG. 6E, a piece of fabric 80 that runs along the entire length of the closure means may be arranged on the inside surface of the closure means in order to protect the skin surface from coming into contact with the inside surface of the closure means. The outer surface of the closure means according to one embodiment, may be covered by two separate pieces of fabric 82A, 82B as shown in FIG. 6A. In particular, as shown in FIG. 6A, when the closure means is attached to the garment pieces of fabric may extend over each side of the closure means rendering it nearly invisible once the closure means is closed. In other words, when the closure means is open, each half of the closure means has a piece of fabric overlaying it such that once the closure means is closed the two pieces of fabric come together fully covering the closure means. Alternatively, as shown in FIG. 6F and outer surface of the closure means may be covered by a single piece of fabric 82C that runs along the entire length of the closure means 70. As shown in the figure, once the closure means is closed, the piece of fabric 83C fully conceals the closure means and thus prevents the outer surface of the closure means from coming into contact with any exposed skin surfaces.

Furthermore, according to one embodiment, as shown in FIGS. 6A and 6E, the piece of fabric or placket 80 that covers the closure means along the inside of the garment can be structured to provide protection against the irritation that can be caused by the closure means ends coming into contact with the neck or chin of the wearer once the closure means is fully closed. In other words, a small pocket 81 can be formed at one end of the closure means by folding one end of the placket over one end of the closure means. More specifically, one end of the piece of fabric or placket 80 can be made to extend beyond the length of the closure means so that it can be folded over the closure means and once attached to the outer surface of the closure means can form a small pocket 81 that can serve as a chin or neck guard 83. Although the closure means may be covered on both sides, as disclosed above, a garment constructed in accordance with another embodiment can be made with only the outside portion of the closure means having the additional piece or pieces of fabric covering it.

According to one embodiment the color of the closure means, such as for example the zipper, can be different from the color of the rest of the garment. In particular, the color of the zipper can be used to distinguish between garments designed for different genders. For example, a blue zipper can be used to specify that the garment is designed for a boy and a pink zipper can be used to specify that the garment is designed for a girl.

Shown in FIG. 6A is yet another feature of the present disclosure which includes a collar 22 attached at the neck opening 24 of the torso covering part of the garment. The collar 22 is stitched along the entire circumference of the neck opening with a seam 84 being on the right side of the garment. Alternatively, the collar may also be attached seamlessly, with all seams being flat lock or on the inside of the garment. This method of attachment provides a greater wear comfort to the wearer by helping to prevent any unnecessary skin irritation that can be caused by the skin surface coming in contact with the seam. Although the collar 22 can have a longitudinal opening that is aligned with the longitudinal elongate opening 28 of the torso covering portion, according to yet another embodiment shown in FIG. 2, the collar 22 can be formed by either attaching a piece of fabric along the full circumferential length of the neck such that no slit is present in the collar or providing a higher neckline that is integrated with the separate torso covering portion of the garment.

Furthermore, in the case when a longitudinal opening 85 is formed in the collar 22 a separate closing means, such as a snap, button, or the like, that would allow for fastening of the collar may also be included. In one particular implementation, a nickel-free snap or a snap made of a material that is least irritable to the skin is used to fasten the collar. In case of a snap, a male and a female parts of the snap are attached at each end of the collar 22, such that when they are engaged a full coverage of the neck is achieved. Alternatively, an additional piece of fabric, with either a male or female portion of the snap attached to it can be sewn to the outer surface of the garment close to the neck and the longitudinal elongate openings. Once engaged with the complement female or male portion of the snap that is attached below the collar at the opposite side of the longitudinal elongate opening, this additional piece of fabric closes a gap that is present between two ends of the collar. However, regardless of the fastening means used or its exact implementation, it is contemplated that the collar is fasten in a place that is least irritating to the skin and thus comfortable to wear.

FIG. 7 shows yet another feature of the garment fabricated in accordance with the present disclosure. As shown in FIG. 7 all components of the garment that necessarily need to be joined, will be sewn together using stitches that are not perceivable by the person wearing the garment. Thus, all seams will be either flat or on the outside of the garment and top stitched or surged to lay down flat. According to one embodiment, a flat lock seam 88 can be use to connect some parts of the garment, such as, for example, Collar, arms, mitts and side seams, while the remaining parts, such as, for example, inseam and the feet covers can be top stitched or surged. The entire garment in accordance with another embodiment of the current disclosure could also be made seamlessly. Because the garment or system of garments will be used repeatedly in both wet and dry environments, seams used to make the garment or system of garments need to be durable. In one embodiment, all the stitching will be done using high durability thread, such as nylon, wooly nylon or poly thread. Also, for extra durability, double stitching may be used. However, it should be understood that any thread or type of seam capable of resisting wear under the specified conditions, and which are also gentle enough when in contact with the skin surfaces can be used. Furthermore, it is also envisioned that the entire garment will be made completely seamless using a v-bed flat knitting machine.

Furthermore, it should be noted that the garment is designed such that the junction areas or the area where the various parts of the garment are joined is minimized. For example, in one such embodiment the entire front and back portion of the garment can be cut out as one piece and joined only at the outer edges of each front and back portion. Alternatively, sleeves can be joined separately to the torso covering portion that is integrated with a leg covering parts of the garment, creating additional attachment area around the shoulder and underarm or a Raglan types of sleeves can be used. Thus, according to this embodiment, two pieces of integrated torso and leg covering portions are joined together, with a first piece of fabric designed to substantially cover an entire front of a torso and legs of a wearer and a second piece of fabric designed to substantially cover an entire back of the torso and legs of the wearer. Also, according to one embodiment as shown in FIG. 6A, an additional piece of fabric 90 can be provided or sewn in around the crotch area in order to provide an adequate space for accommodating, for example, a diaper when the garment is used for infants. The additional space afforded by the piece of fabric 90 is useful as the overall snug fitting design may lead to an overly tight fit in situations when diapers are used under the garment. Thus, for example, an oval shaped piece of fabric can be used to join the first and the second integrated torso and leg covering portions in around the crotch area thereby providing the needed additional space.

Also, it is yet another feature of the current disclosure, that in those instances where labels need to be used with the garment, they will be on the outside of the garment and could be easy to tear off. Alternatively, all the labels will be PVC free, heat press labels placed on the outside of the garment.

Although the disclosed garment may be used to cover the entire body of the wearer as discussed above, a garment 15 designed to cover only a specific body part that is most likely to be affected by any of the previously discussed skin disorders, may also be used. As shown in FIG. 8, one such garment may be designed to cover only, for example, knees, elbows, or hands. In particular, depending on the area that is to be treated, a partial garment can be constructed from a piece of fabric that is sewn into a tubular form that fits closely over the selected body part, along with an immediately adjacent areas. More specifically, in addition to covering, for example, an elbow of a wearer, the garment can extend from the middle of the upper arm of the wearer to the middle of the lower arm of the wearer with the middle of the upper arm being approximately halfway between a shoulder and an elbow of the wearer and the middle of the lower arm being approximately halfway between the elbow and a wrist of the wearer. Alternatively, the partial garment may extend from the middle of the upper arm at one end and include a hand covering portion and/or glove at the other end.

It should be understood that garment or system of garments disclosed are designed in such a manners as to provide maximum comfort to the individual wearing them and they are both attractive and functional. Changes may be made to the above garment or system of garments without departing from the scope hereof. It should thus be noted that the matter contained in the above description or shown in the accompanying figures should be interpreted as illustrative and not in a limiting sense.

1. A garment for treatment of atopic dermatitis comprising:
a torso covering portion supporting a pair of leg covering portions and a pair of arm covering portions, wherein the torso covering portion includes a longitudinal elongate opening along the torso covering portion, the longitudinal elongate opening extending through substantially an entire length of the torso covering portion between a neckline of the garment and terminating at around a crotch area, and wherein the torso covering portion, the leg covering portions and the arm covering portions are made from a fabric capable of conforming to a body of a wearer such that the garment remains in substantially direct contact with the skin surface of the wearer, the fabric capable of moisturizing the skin surface and being optimized for moisture absorption when the fabric is being wetted and for a prolonged moisture release when the fabric is placed against the surface of the skin.

2. The garment of claim 1, wherein the junction areas between the torso covering portion the leg covering portions and the arm covering portions of the garment are minimized by forming some of the torso covering portion, the leg covering portions and the arm covering portions from a single piece of fabric in order to prevent any unnecessary skin irritation that can be caused by the skin coming into contact with the junction areas.

3. The garment of claim 1 further including a pair of hand covering portions, each of the hand covering portions and the arm covering portions forming an extendable sleeve having a cuff assembly at a distal end including a foldable cuff panel, the foldable cuff panel being formed from a piece of fabric having a first longitudinal edge and a second longitudinal edge and a first lateral edge and a second lateral edge, the piece of fabric being attached to an outer surface of the extendable sleeve along the first and the second longitudinal edges and the first lateral edge, the first lateral edge attached to a lower edge of the arm covering portion along a part of the circumference of the lower edge of the arm covering portion, with the second lateral edge of the foldable cuff panel being a free edge longitudinally displaced from the first lateral edge, the free edge capable of being pulled over the fingers of a wearer when the cuff assembly is in use thereby forming a fold-over hand covering, and wherein the first longitudinal edge, the second longitudinal edge and the first lateral edge are all attached to the outer surface of the extendable sleeves with seams being visible on the outside of the extendable sleeve when the extendable sleeve is in an unfolded configuration.

4. The garment of claim 3 wherein the foldable cuff panel is formed from a rectangularly shaped piece of fabric covering a half of the circumference of the sleeve and wherein seams used to attach the first and the second longitudinal edges and the first lateral edge to the outer surface of the extendable sleeve are all different.

5. The garment of claim 3 wherein the piece of fabric forming the foldable cuff panel is attached to the outer surface of the extendable sleeve, on a palmar side of a hand.

6. The garment of claim 3 wherein the piece of fabric forming the foldable cuff panel is attached to the outer surface of the extendable sleeve, on a dorsal side of a hand.

7. The garment of claim 3 wherein the piece of fabric is a two-ply fabric, having an inner ply and an outer ply, with both the inner and the outer ply being continuously knit to each other along the second lateral edge.

8. The garment of claim 3 further comprising a glove attached to an inside surface of each of the extendable sleeves at a wrist area along a part of the circumference of the extendable sleeve.

9. The garment of claim 1 wherein the longitudinal elongate opening includes a closure means and a piece of fabric extending along an entire length of the closure means, the piece of fabric forming a barrier between the closure means and the skin surface of the wearer in order to prevent the closure means from coming into contact with the skin surface of the wearer when the garment is in use.

10. The garment of claim 1 wherein the fabric is a microfiber fabric.

11. The body garment of claim 10 wherein the feet covering portions have flat seams along an ankle area and around an outer parameter of a foot.

12. The body garment of claim 1 wherein all seams used to join various parts of the garment are on the outside of the garment.

13. A full-body garment for treatment of atopic dermatitis comprising:
a close fitting torso covering portion provided with a pair of a close fitting pair of leg covering portions, the close fitting torso covering portion and the close fitting pair of leg covering portions formed from a first piece of fabric covering substantially an entire front of the torso and legs of a wearer, the first piece of fabric having a longitudinal elongate opening extending through substantially an entire length of the torso covering portion between a neckline of the garment and terminating at around a crotch area, the longitudinal elongate opening including a closure means and a third piece of fabric extending along the entire length of the closure means, the third piece of fabric forming a barrier between the closure means and a skin surface of the wearer when the garment is worn, and a second piece of fabric covering substantially an entire back of the torso and legs of the wearer, the first and the second pieces of fabric being joined together by a seam along an outer parameter of the first and second pieces of fabric at the side of the garment and along the inner side of the leg covering portions;
a pair of close fitting extendable sleeves, each extendable sleeve formed from a piece of fabric sewn into a tubular form with a seam running along the length of the extendable sleeve and having a cuff assembly at a distal end, the cuff assembly having a foldable cuff panel formed from a piece of fabric having a first longitudinal edge and a second longitudinal edge extending along the extendable sleeve and a first lateral edge and a second lateral edge traversing the sleeve, the cuff panel covering a half of the circumference of the sleeve, with one of the lateral edges sewn along lower edge of the extendable sleeve and the second lateral edge being a free edge longitudinally displaced from the first lateral edge, the close fitting extendable sleeve having a length that is substantially longer than the arm length of a wearer and having a first open position and a second folded position, the first open position characterized by having the cuff assembly extend to the wrist of the wearer such that the hand of the wearer extends beyond the end of the cuff assembly and the second folded position covering the hand of the wearer by pulling the free edge of the cuff panel over the fingers of the wearer so as to provide a fold over hand covering, the extendable sleeve connected to both the first and the second pieces of fabric by a seam along the circumferential parameter of the proximal end of the extendable sleeve; and
a pair of feet covering portions attached at distal ends of the leg covering portions, each of the feet covering portions formed by an instep covering portion and a sole covering portion, the instep covering portion attached to the sole covering portion along a substantial portion of their respective parameters, the instep covering portion further attached by a seam to the distal end of the leg covering portion formed from the first piece of fabric and the sole covering portion further attached by a seam to the distal end of the leg covering portion formed from the second piece of fabric, wherein the close fitting torso covering portion with a pair of a close fitting pair of leg covering portions, the a pair of close fitting extendable sleeves and the pair of feet covering portions are made from a fabric capable of conforming to a body of a wearer such that the garment remains in substantially direct contact with the skin surface of the wearer, the fabric capable of moisturizing into the skin surface and being optimized for moisture absorption when the fabric is being wetted and for a prolonged moisture release when the fabric is placed against the surface of the skin.

14. The full body garment of claim 13 wherein the closure means includes a zipper and the third piece of fabric extends beyond the length of the zipper and when fold over the end of the zipper at the neckline provides a protection from the zipper rubbing against the chin of the wearer.

15. The full body garment of claim 13 wherein a gusset panel is sewn in between the first and the second pieces of fabric around the crotch area.

16. A garment for treatment of atopic dermatitis comprising:
a torso covering portion;
a pair of leg covering portions;
a pair of arm covering portions;
wherein the torso covering portion, the pair of leg covering portions and the pair of arm covering portions are made from a fabric capable of conforming to a body of a wearer such that the garment remains in substantially direct contact with the skin surface of the wearer, the fabric capable of supplying moisture to the skin surface and being optimized for moisture absorption when the fabric is being wetted and for a prolonged moisture release when the fabric is placed against the surface of the skin and the torso covering portion and the leg covering portions are separate.

17. A garment for treatment of atopic dermatitis comprising:
a close fitting arm covering portion having a tubular shape, the close fitting arm covering portion designed to cover a portion of an arm of a wearer from about the middle of the upper arm to about the middle of the lower arm, the close fitting arm covering portion being made from a fabric capable of conforming to the arm of the wearer such that the arm covering portion remains in substantially direct contact with the skin surface of the arm of the wearer, the fabric capable of supplying moisture to the skin surface and being optimized for moisture absorption when the fabric is being wetted and for a prolonged moisture release when the fabric is placed against the surface of the skin.
